(19) United States
(12) Patent Application Publication
(10) Pub. No.: US 2010/0249449 A1
(43) Pub. Date: Sep. 30, 2010

(54) METHOD FOR AMIDATING OF NITRILE IN THE PRESENCE OF SULFURIC ACID

(75) Inventors: Lorenza Sartorelli, Ober-Ramstadt (DE); Andreas Perl, Bobenheim-Roxheim (DE); Udo Gropp, Bad Endorf (DE); Arndt Selbach, Dirmstein (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: Evonik Roehm GmbH, Darmstadt (DE)

(21) Appl. No.: 12/738,094

(22) PCT Filed: Aug. 15, 2008
(86) PCT No.: PCT/EP08/60731

§ 371 Date: Apr. 15, 2010

(30) Foreign Application Priority Data
Oct. 18, 2007 (DE) 102007050284.4

(51) Int. Cl. C07C 231/06; C07C 67/22

(52) U.S. Cl. 560/215; 564/124; 564/130

(57) ABSTRACT

The present invention relates to a process for preparing carboxamides by amidating nitriles in the presence of sulphuric acid, wherein the reaction is performed in a Taylor reactor. The process enables a simple and inexpensive preparation of these compounds. The present invention further provides processes for preparing (meth)acrylamides and alkyl(meth)acrylates, which comprise an inventive amidation reaction.

### FIELD OF THE INVENTION

The present invention relates to processes for amidating nitriles in the presence of sulphuric acid. The present invention further describes processes for preparing (meth)acrylamides and alkyl(meth)acrylates, in which a cyanohydrin is amidated.

The preparation of carboxamides by the amidation of nitriles is a widely employed process. For example, such an amidation takes place as an important intermediate step in the preparation of methyl methacrylate by the ACH process, and uses large amounts of sulphuric acid.

### STATE OF THE ART

A process representative of such a process is, for example, that described in U.S. Pat. No. 4,529,816, according to which the ACH amidation is performed at temperatures around 100° C. with a molar ratio of ACH:H₂SO₄ of about 1:1.3 to 1:1.8. Relevant process steps are: a) amidation; b) conversion; and c) esterification.

In the amidation, the main products obtained from the reaction are SIBA=sulphoxy-alpha-hydroxyisobutyramide hydrogensulphate and MAA*H₂SO₄=methacrylamide hydrogensulphate as a solution in excess sulphuric acid.

In addition, in a typical amidation solution, HIBA*H₂SO₄=alpha-hydroxyisobutyramide hydrogensulphate is also obtained with a yield based on ACH of <5%. In the case of more or less complete ACH conversion, this amidation process which is in principle quite selective proceeds with a yield (=sum of the intermediates described) of approx. 96%-97%.

The by-products formed in this step are thus, however, already not inconsiderable amounts of carbon monoxide, acetone, sulphonation products of acetone and cyclocondensation products of acetone with various intermediates.

The aim of the conversion is the very substantially complete conversion of SIBA and HIBA to MAA, which proceeds with β-elimination of sulphuric acid (in excess sulphuric acid as the solvent).

In the process step of conversion, the (anhydrous) solution of HIBA, SIBA and MAA in sulphuric acid (each of which is present as the hydrogensulphate) are then converted in the conversion at high temperatures between 140° C.-160° C. and short residence times of about 10 min or less.

The conversion mixture of this procedure is characterized by a high excess of sulphuric acid and the presence of the MASA*H₂SO₄ main product with a concentration in the solution of about 30% by weight-35% by weight (according to the sulphuric acid excess used).

The process described in U.S. Pat. No. 4,529,816 has the disadvantage that far greater than stoichiometric amounts of sulphuric acid have to be used. In addition, tar-like, solid condensation products separate out of the ammonium hydrogensulphate- and sulphuric acid-containing process acid which is regenerated in the sulphuric acid contact plant, and prevent trouble-free conveying of the process acid and have to be eliminated with a considerable level of cost and inconvenience.

Owing to the drastic yield losses in the above-described process from U.S. Pat. No. 4,529,816, there are some proposals to amidate and hydrolyse ACH in the presence of water, in which case the hydroxyl function is maintained in the molecular unit at least in the first steps of the reaction.

Depending on whether they are carried out in the presence of or without methanol, these proposals for an alternative amidation in the presence of water lead either to the formation of methyl hydroxyisobutyrate (=MHIB) or to the formation of 2-hydroxyisobutyrate (=HIBA).

A further alternative for the preparation of esters of alpha-hydroxyisobutyric acid, especially methyl alpha-hydroxyisobutyrate, proceeding from ACH is described in JP Hei-4-193845. In JP Hei-4-193845, ACH is first amidated with 0.8 to 1.25 equivalents of sulphuric acid in the presence of less than 0.8 equivalent of water below 60° C. and then reacted at temperatures of greater than (>) 55° C. with more than 1.2 equivalents of alcohol, especially methanol, to give MHIB or corresponding esters. There is no mention here of the presence of viscosity-reducing media which are stable toward the reaction matrix.

The disadvantages and problems of this process are the industrial implementation as a result of exceptional viscosity formation at the end of the reaction.

It is also known that hydroxyisobutyric acid can be prepared proceeding from acetone cyanohydrin (ACH) by performing the hydrolysis of the nitrile function in the presence of mineral acids (see U.S. Pat. No. 222,989; J. Brit. Chem. Soc. (1930); Chem. Ber. 72 (1939), 800].

A process representative of such a process is, for example, Japanese patent publication Sho 63-61932, in which ACH is hydrolysed to hydroxyisobutyric acid in a two-stage process. In this process, ACH is first converted in the presence of 0.2-1.0 mol of water and 0.5-2 equivalents of sulphuric acid, which forms the corresponding amide salts.

As early as in this step, in the case of use of small water and sulphuric acid concentrations which are needed to obtain good yields, short reaction times and small amounts of waste process acid, massive problems occur with the stirrability of the amidation mixture as a result of high viscosity of the reaction mixtures, especially towards the end of the reaction time.

When the molar amount of water is increased to ensure a low viscosity, the reaction is slowed drastically and side reactions occur, especially the fragmentation of ACH into the acetone and hydrocyanic acid reactants, which react further to give conversion products under the reaction conditions.

According to the statements of the problem in Japanese patent publication SHO 63-61932, an increase in the temperature also allows the viscosity of the reaction mixture to be controlled, and the corresponding reaction mixtures become stirrable as a result of the falling viscosity, but the side reactions increase drastically here too even at moderate temperatures, which is ultimately manifested in only moderate yields (see comparative examples).

When low temperatures<50° C. are employed, which would ensure a selective reaction, the increase in the concentration of amide salts which are sparingly soluble under the reaction conditions results, toward the end of the reaction time, first in the formation of a suspension which is difficult to stir and finally in the complete solidification of the reaction mixture.

In the second step of Japanese patent publication SHO 63-61932, water is added to the amidation solution and hydrolysed at temperatures higher than the amidation temperature, which forms hydroxyisobutyric acid from the amide salts formed after the amidation with release of ammonium hydrogensulphate.

As well as the selective preparation of the HIBA target product in the reaction, another essential factor for the economic viability of an industrial process is the isolation of the reaction matrix and/or the removal of HIBA from the remaining process acid.

JP Sho 57-131736, method for isolating alpha-hydroxyisobutyric acid (=HIBA), addresses this problem by treating the reaction solution which is obtained after the reaction between acetone cyanohydrin, sulphuric acid and water by hydrolytic cleavage and comprises alpha-hydroxyisobutyric acid and acidic ammonium hydrogensulphate with an extractant, which transfers the hydroxyisobutyric acid into the extractant while the acidic ammonium sulphate remains in the aqueous phase.

In this process, before the extraction, the free sulphuric acid which is still present in the reaction medium is neutralized by treating with an alkali medium in order to increase the degree of extraction of HIBA into the organic extraction phase.

The necessary neutralization is associated with considerable additional expenditure of aminic or mineral base and hence with considerable waste amounts of corresponding salts, which cannot be disposed of in an ecologically responsible and economically viable manner.

Furthermore, publication DE 10 2004 006 826 describes a process for preparing methacrylic acid and corresponding esters proceeding from cyanohydrin.

According to this publication, acetone cyanohydrin is reacted at temperatures below 80° C. with not more than 1.5 equivalents of sulphuric acid in the presence of 0.05-1.0 equivalent of water in the presence of a polar solvent which is inert under the reaction conditions to form a readily stirrable solution of the corresponding amide sulphates in the inert polar solvent. Subsequently, water is added to the mixture thus obtained, and the inert solvent is removed.

Subsequently, hydroxyisobutyric acid is removed from the aqueous ammonium hydrogensulphate solution by extraction with a suitable extractant.

The process detailed in DE 10 2004 006 826 leads to good yields.

However, a multitude of steps is required, which adversely affects the economic viability of the overall process.

### PROBLEM AND SOLUTION

In view of the prior art, it was thus an object of the present invention to provide a process for preparing carboxamides by amidating nitriles in the presence of sulphuric acid, in which the product can be obtained in a very economically viable manner. More particularly, the process should be performable with a minimum excess of sulphuric acid. It was therefore a further object of the present invention to provide a process of the aforementioned type, which can be carried out without addition of high amounts of inert solvents or water. We do not use any water or other solvents. Furthermore, the carboxamide obtained should contain only very small amounts of by-products.

It was a further object of the invention to provide a process in which the carboxamide can be obtained very selectively.

It was a further object of the present invention to provide processes for preparing carboxamides by amidating nitriles in the presence of sulphuric acid, which can be performed simply and inexpensively. At the same time, the product should, as far as possible, be obtained in high yields and, viewed overall, with a low energy consumption.

These objects and further objects which are not stated explicitly but which can be derived or discerned immediately from the connections discussed herein by way of introduction are achieved by processes having all features of claim 1. Appropriate modifications of the process according to the invention are protected in the dependent claims referring back to claim 1.

The present invention accordingly provides a process for preparing carboxamides by amidating nitriles in the presence of sulphuric acid, which is characterized in that the reaction is performed in a Taylor reactor.

This makes it possible, in an unforeseeable manner, to provide a process for preparing carboxamides in which the product is obtained in a very economically viable manner. Surprisingly, the resulting product contains only very small amounts of by-products. Furthermore, the process according to the invention enables particularly selective preparation of carboxamides.

Moreover, the process according to the invention can be performed in a simple and inexpensive manner, and the product can be obtained in high yields and, viewed overall, with low energy consumption.

In addition, the process according to the invention can achieve a particularly economically viable operating mode of the production plant, and simple and inexpensive adjustment of the production rate to the target rate desired in each case can be achieved, without complicated adjustment of the production conditions, especially of the reaction temperature and of the pressure at which the reaction takes place being necessary, such that further, surprising advantages can be achieved.

By virtue of the inventive measures, it is surprisingly possible to achieve particularly simple and rapid homogenization of the starting mixture in a Taylor reactor with simultaneously rapid adjustment of the thermal conditions to predefined values.

The process according to the invention enables the efficient preparation of carboxamides. In this process, nitriles in particular are used, which generally have groups of the formula —CN. Carboxamides include at least one group of the formula —CONH₂. These compounds are known in the technical field and are described, for example, in Römpp Chemie Lexikon, 2nd edition on CD-ROM.

The reactants used may especially be aliphatic or cycloaliphatic nitriles, saturated or unsaturated nitriles and aromatic and heterocyclic nitriles. The nitriles to be used as reactants may have one, two or more nitrile groups. In addition, it is also possible to use nitriles which have heteroatoms, especially halogen atoms such as chlorine, bromine, fluorine, oxygen atoms, sulphur atoms and/or nitrogen atoms in the aromatic or aliphatic radical. Preferably, particularly suitable nitriles include 2 to 100, preferably 3 to 20 and most preferably 3 to 5 carbon atoms.

The aliphatic nitriles which each have a saturated or unsaturated hydrocarbon group include acetonitrile, propionitrile, butyronitrile, isobutyronitrile, valeronitrile, isovaleronitrile, capronitrile and other saturated mononitriles; malonitrile, succinonitrile, glutaronitrile, adiponitrile and other saturated dinitriles; α-aminopropionitrile, α-aminomethylthiobutyronitrile, α-aminobutyronitrile, aminoacetonitrile and other α-aminonitriles; cyanoacetic acid and other nitriles having one carboxyl group each; amino-3-propionitrile and other β-aminonitriles; acrylonitrile, methacrylonitrile, allyl cyanide, crotononitrile or other unsaturated nitriles, and cyclopentanecarbonitrile and cyclohexanecarbonitrile and other alicyclic nitriles.

The aromatic nitriles include benzonitrile, o-, m- and p-chlorobenzonitrile, o-, m- and p-fluorobenzonitrile, o-, m- and p-nitrobenzonitrile, p-aminobenzonitrile, 4-cyanophenol, o-, m- and p-tolunitrile, 2,4-dichlorobenzonitrile, 2,6-dichlorobenzonitrile, 2,6-difluorobenzonitrile, anisonitrile, α-naphthonitrile, β-naphthonitrile and other aromatic mononitriles: phthalonitrile, isophthalonitrile, terephthalonitrile and other aromatic dinitriles; benzyl cyanide, cinnamoylnitrile, phenylacetonitrile, mandelonitrile, p-hydroxyphenylacetonitrile, p-hydroxyphenylpropionitrile, p-methoxyphenylacetonitrile and other nitriles which each have an aralkyl group.

The heterocyclic nitriles include especially nitrile compounds which each have at least one heterocyclic group which contains a 5- or 6-membered ring and has at least one atom which is selected from the group consisting of a nitrogen atom, an oxygen atom and a sulphur atom as a heteroatom, for example 2-thiophenecarbonitrile, 2-furonitrile and other nitriles which each have a sulphur atom or an oxygen atom as a heteroatom; 2-cyanopyridine, 3-cyanopyridine, 4-cyanopyridine, cyanopyrazine and other nitriles which each contain a nitrogen atom as a heteroatom; 5-cyanoindole and other fused heterocycles; cyanopiperidine, cyanopiperazine and other hydrogenated heterocyclic nitriles and fused heterocyclic nitriles.

The particularly preferred nitriles include especially α-hydroxynitriles (cyanohydrins), for example hydroxyacetonitrile, 2-hydroxy-4-methylthiobutyronitrile, α-hydroxy-γ-methylthiobutyronitrile (4-methylthio-2-hydroxybutyronitrile), 2-hydroxypropionitrile (lactonitrile) and 2-hydroxy-2-methylpropionitrile (acetone cyanohydrin), particular preference being given to acetone cyanohydrin.

Of particular interest are especially processes in which the nitrile, especially acetone cyanohydrin (ACH), is converted in an excess of sulphuric acid. The molar ratio of sulphuric acid to nitrile is preferably in the range from 1.2:1 to 3:1, more preferably in the range from 1.2:1 to 1.8:1, and the reaction can also be carried out in a plurality of steps. In this case, the molar ratios in the individual steps can be varied. Compared to the customary reactions, for example in loop reactors, the reaction can be carried out at a significantly lower content of sulphuric acid. This reduces the waste (cleavage acid), and so the reaction can be effected in a more economically viable and environmentally protective manner.

These data are based on 100% by weight of sulphuric acid, which may also contain disulphuric acid (H₂S₂O₇). Small amounts of water may likewise be present in the reaction mixture, although preference is given to processes in which concentrated sulphuric acid is used, which has a minimum content of 95% by weight, more preferably 98% by weight and most preferably 100% (oleum) of sulphuric acid.

In addition, the reaction mixture may comprise customary additives, for example phenothiazine or other stabilizers.

Taylor reactors, which serve to convert substances under the conditions of Taylor vortex flow, are known. They consist of two coaxial concentric cylinders of which generally the outer cylinder is fixed and the inner cylinder rotates. The reaction chamber used is the volume which is formed by the gap of the cylinder. With increasing angular velocity ω of the inner cylinder, a number of different flow types occur, which are characterized by a dimensionless index, the so-called Taylor number Tₐ. In addition to the angular velocity of the stirrer, the Taylor number is also dependent on the kinematic viscosity ν of the fluid in the gap and on the geometric parameters, the outer radius of the inner cylinder rᵢ, the inner radius of the outer cylinder rₐ and the gap width d, the difference of the two radii, according to the following formula:

$${T}_{a} = \frac{ω \cdot {r}_{i} \cdot d}{v} \sqrt{\frac{d}{{r}_{i}}}$$

where
d: gap width (d=rₐ−rᵢ)
rₐ: outer radius of the inner cylinder
rᵢ: inner radius of the outer cylinder
ν: kinematic viscosity of the fluid in the gap
ω: angular velocity of the stirrer (inner cylinder) s⁽⁻¹⁾

In addition, the axial Reynolds number Reₐₓ may serve to describe the flow conditions in a Taylor reactor, which is likewise a dimensionless index. The axial Reynolds number Reₐₓ is calculated from the formula:

$${Re}_{ax} = \frac{{U}_{ax} \cdot d}{v}$$

where
d: gap width (d=rₐ−rᵢ)
rₐ: outer radius of the inner cylinder
rᵢ: inner radius of the outer cylinder
ν: kinematic viscosity of the fluid in the gap
Uₐₓ: axial flow velocity

In a particular embodiment of the present process, the amidation can be carried out at a Taylor number in the range from 10 to 3000, more preferably in the range from 20 to 2500 and most preferably in the range from 50 to 2000. The axial Reynolds number in the amidation reaction may, for example, be in the range from 0.1 to 100 and more preferably in the range from 1 to 50.

As well as Taylor reactors with two coaxial concentric cylinders, it is also possible to use corresponding reactors which have, for example, an outer reactor wall and a concentric or eccentric rotor disposed therein, as described by way of example in DE 199 60 389 or WO 2004/039491.

In these reactors, the values detailed above for the Taylor number and the axial Reynolds number are calculated correspondingly, and the flow through the Taylor reactor may be in either direction.

The reaction can be effected under elevated pressure or reduced pressure. In a particularly appropriate modification of the present invention, the amidation can be carried out at a pressure in the range from 200 mbar to 5 bar and more preferably in the range from 500 mbar to 2 bar.

The reaction temperature may, especially as a function of the pressure, likewise be within a wide range. In a preferred embodiment of the present invention, the amidation is effected preferably at a temperature in the range from 50° C. to 150° C., more preferably in the range from 60° C. to 140° C. and most preferably 80° C. to 100° C.

The process of the present invention can be performed continuously. Appropriately, the residence times may generally be in the range from 50 seconds to 5 hours, preferably 5 minutes to 3 hours and most preferably 15 minutes to 2 hours. In this context, the residence time can be varied by means of inlets and outlets at various points in the reactor. This embodiment of the present invention enables a particularly simple and appropriate adjustment of the production rate to given ordered amounts, without any need for the pressure and the temperature to be altered in a complicated manner.

It is surprisingly possible by virtue of the inventive measures to provide a process which can also be performed with a reaction mixture which has a high viscosity. For instance, the viscosity of the reaction mixture may preferably be 5 to 1000 mPa*s, more preferably 10 to 500 mPa*s and most preferably 20 to 200 mPa*s.

It is more preferably possible especially to amidate cyanohydrins, for example acetone cyanohydrin, preferably to obtain sulphoxy-alpha-hydroxyisobutyramide hydrogensulphate (SIBA) and/or alpha-hydroxyisobutyramide (HIBA), which can be used especially to prepare (meth)acrylamide. Accordingly, the present invention also provides a process for preparing (meth)acrylamide which comprises an inventive amidation in a Taylor reactor.

Particular advantages can surprisingly be achieved by performing the conversion of the hydroxycarboxamide to the (meth)acrylamide in a Taylor reactor. Appropriately, the dehydration can be performed, for example, at a Taylor number in the range from 100 to 10 000, more preferably in the range from 500 to 5000. The axial Reynolds number at which the reaction of the hydroxycarboxamide is carried out may preferably be in the range from 10 to 500, more preferably in the range from 15 to 300.

The pressure at which the dehydration of the hydroxycarboxamide is carried out is not critical per se. Accordingly, this reaction can be performed under elevated pressure or reduced pressure. In general, the dehydration can be effected at a pressure in the range from 200 mbar to 5 bar, more preferably in the range from 500 mbar to 2 bar.

The reaction temperature may, especially depending on the pressure, likewise be within a wide range. In a preferred embodiment of the present invention, the dehydration is effected preferably at a temperature in the range from 50° C. to 220° C., more preferably in the range from 70° C. to 200° C. and most preferably 120 to 180° C.

The dehydration process of the present invention can be performed continuously. Appropriately, the residence times may generally be in the range from 30 seconds to 3 hours, preferably in the range from 1 minute to 2 hours and most preferably in the range from 10 to 60 minutes.

The performance of the dehydration process in a Taylor reactor enables surprising advantages. By virtue of inlets and/or outlets at various points in the reactor, the residence time can be varied in a particularly simple manner via the reactor volume, without any need for complicated optimization of pressure and temperature. This makes it possible to undertake simple adaption of the production rate to a given target rate. In addition, different heating zones can be selected, which are then switched on or off as required. This variation gives rise to the possibility of enhancing the yield.

The compounds detailed above in many cases serve as an intermediate for preparing alkyl(meth)acrylates. Accordingly, the process steps detailed above may serve to prepare alkyl(meth)acrylates, and so the present invention further provides a process for preparing alkyl(meth)acrylates which includes an inventive amidation of a cyanohydrin.

This reaction is described, inter alia, in U.S. Pat. No. 4,529,816, wherein the carboxamide is reacted with an alcohol which comprises preferably 1-10 carbon atoms and more preferably 1 to 5 carbon atoms. Preferred alcohols include methanol, ethanol, propanol, butanol, especially n-butanol and 2-methyl-1-propanol, pentanol, hexanol, heptanol, 2-ethylhexanol, octanol, nonanol and decanol.

The alcohol used is more preferably methanol and/or ethanol, very particular preference being given to methanol. The reaction of carboxamides with alcohols to obtain carboxylic esters is common knowledge. This reaction preferably takes place in the presence of sulphuric acid.

The molar ratio of α-hydroxycarboxamide and/or methacrylamide to alcohol, for example α-hydroxyisobutyramide and/or methacrylamide to methanol, is not critical per se, and is preferably in the range from 3:1 to 1:20, more preferably in the range from 1:10 to 1:1.

The reaction temperature may likewise be within wide ranges, the reaction rate generally increasing with increasing temperature. The upper temperature limit arises generally from the boiling point of the alcohol used.

The reaction temperature is preferably in the range of 40° C.-200° C., more preferably 130° C.-180° C. According to the reaction temperature, the reaction can be carried out under elevated pressure or reduced pressure. This reaction is preferably carried out within a pressure range from 200 mbar to 5 bar, more preferably in the range from 500 mbar to 2 bar.

In a particular embodiment, the aforementioned reactions, especially the conversion of (meth)acrylamides to alkyl(meth)acrylate, can be performed in the presence of polymerization inhibitors. These compounds, for example hydroquinones, hydroquinone ethers such as hydroquinone monomethyl ether or di-tert-butylpyrocatechol, phenothiazine, 4-hydroxy-2,2,6,6-tetramethylpiperidine 1-oxyl, N,N′-diphenyl-p-phenylenediamine, methylene blue or sterically hindered phenols, are widely known in the technical field. These compounds may be used individually or in the form of mixtures and are generally commercially available. The action of the stabilizers usually consists in their action as free-radical scavengers for the free radicals which occur in the course of polymerization.

For further details, reference is made to the current technical literature, especially to Römpp-Lexikon Chemie; editors: J. Falbe, M. Regitz; Stuttgart, N.Y.; 10th edition (1996); under “Antioxidants” and the literature references cited at this point. Based on the weight of the overall reaction mixture, the proportion of the inhibitors, individually or as a mixture, may generally be 0.01-0.5% (wt/wt).

1. A process for preparing carboxamides by amidating nitriles in the presence of sulphuric acid, wherein the reaction is performed in a Taylor reactor.

2. The process according to claim 1, wherein the nitrile is a cyanohydrin.

3. The process according to claim 2, wherein the cyanohydrin is acetone cyanohydrin.

4. The process according to claim 1, wherein the process is performed at a Taylor number in the range from 100 to 10 000.

5. The process according to claim 4, wherein the process is performed at a Taylor number in the range from 500 to 5000.

6. The process according to claim 1, wherein the process is performed at an axial Reynolds number in the range from 0.1 to 100.

7. The process according to claim 1, wherein the molar ratio of sulphuric acid to nitrile is in the range from 1.2:1 to 3:1.

8. The process according to claim 1, wherein the process is performed at a temperature in the range from 50° C. to 150° C.

9. The process according to claim 1, wherein the process is performed continuously.

10. The process according to claim 1, wherein the residence time is in the range from 5 minutes to 3 hours.

11. The process according to claim 1, wherein the viscosity of the reaction mixture is in the range from 5 mPa*s to 1000 mPa*s.

12. A process for preparing (meth)acrylamides, wherein the process includes an amidation of a cyanohydrin by a process according to claim 1.

13. The process according to claim 12, wherein the conversion of the carboxamide to the (meth)acrylamide is performed in a Taylor reactor.

14. The process according to claim 13, wherein the process is performed at a Taylor number in the range from 100 to 10 000.

15. The process according to claim 13, wherein the process is performed at an axial Reynolds number in the range from 10 to 500.

16. The process according to claim 12, wherein sulphoxy-alpha-hydroxyisobutyramide hydrogensulphate (SIBA) is converted to methacrylamide.

17. The process according to claim 12, wherein the process is performed at a temperature in the range from 70° C. to 180° C.

18. The process according to claim 12, wherein the process is performed continuously.

19. The process according to claim 12, wherein the residence time is in the range from 1 minute to 2 hours.

20. A process for preparing alkyl(meth)acrylates, wherein the process includes an amidation of a cyanohydrin by a process according to claim 1.

21. The process according to claim 20, wherein methacrylamide is reacted with an alcohol having 1 to 5 carbon atoms in the presence of sulphuric acid.

22. The process according to claim 20, wherein methyl methacrylate is prepared.
